(19) United States
(12) Patent Application Publication
(10) Pub. No.: US 2005/0032089 A1
(43) Pub. Date: Feb. 10, 2005

(54) Sample preparation for colorimetric and fluorescent assays as implemented on optical analysis discs

(76) Inventors: Brigitte Chau Phan, Irvine, CA (US); Amethyst Hoang Lam, Irvine, CA (US); Shih-Li Lo, Fullerton, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/793,335

(22) Filed: Mar. 4, 2004

Related U.S. Application Data
(60) Provisional application No. 60/452,313, filed on Mar. 5, 2003.

(51) Int. Cl. B05D 3/00; C12Q 1/68; C12M 1/34

(52) U.S. Cl. 435/6; 435/287.2; 427/2.11

(57) ABSTRACT

A wide variety of current diagnostic and other biochemical tests employ a substance, such as a chromagen, that undergoes a detectable color development or change of fluorescent emission in the presence of the analyte of interest. The intensity of the color or fluorescence developed may be time dependent and proportional to the concentration of the analyte of interest. Systems, methods, and components usable for quantifying the concentration of an analyte of interest in a biological sample on optical biodiscs are disclosed herein. Analytes may include, for example, glucose, cholesterol, and triglycerides. In one embodiment, reagents are immobilized on the optical disc prior to the assay.

### RELATED APPLICATIONS

This application claims priority under 35 U.S.C. §119(e) to U.S. Provisional Application Ser. No. 60/452,313, filed Mar. 5, 2003, the disclosure of which is incorporated by reference herein in its entirety.

### BACKGROUND OF THE INVENTION

1. Field of the Invention

This invention relates in general to assays and, in particular, colorimetric and fluorescent assays. More specifically, but without restriction to the particular embodiments hereinafter described in accordance with the best mode of practice, this invention relates to sample preparation for colorimetric and fluorescent assays as performed on optical analysis discs.

2. Description of the Related Art

Detection and quantification of analytes in body fluids, such as blood, may be important for diagnosis of diseases, elucidation of the pathogenesis, and monitoring the response to drug treatment. Traditionally, diagnostic assays are performed in laboratories by trained technicians using complex apparatus. Performing these assays is usually time-consuming and costly. Thus, there is a significant need to make diagnostic assays and forensic assays faster and more local to the end-user. Ideally, clinicians, patients, investigators, the military, other health care personnel, and consumers should be able to test themselves for the presence of certain risk factors or disease indicators in their systems, and to test for the presence of certain biological material at a crime scene or on a battlefield. At present, there are a number of medical diagnostic, silicon-based, devices with nucleic acids and/or proteins attached thereto that are commercially available or under development. These chips are not for use by the end-user, or for use by persons or entities lacking very specialized expertise and expensive equipment.

Commonly assigned U.S. Pat. No. 6,030,581 entitled “Laboratory in a Disk” issued Feb. 29, 2000 (the '581 patent) is hereby incorporated by reference in its entirety. The '581 patent discloses an apparatus that includes an optical disc, adapted to be read by an optical reader, which has a sector having a substantially self-contained assay system useful for localizing and detecting an analyte suspected of being in a sample. U.S. Pat. No. 5,993,665, issued Nov. 30, 1999 (the '665 patent) entitled “Quantitative Cell Analysis Methods Employing Magnetic Separation” discloses analysis of biological specimens in a fluid medium where the specimens are rendered magnetically responsive by immuno-specific binding with ferromagnetic colloid. The '665 patent is hereby incorporated by reference in its entirety.

### SUMMARY OF THE INVENTION

The present invention relates to performing colorimetric and fluorescent assays on an optical analysis disc. The invention includes methods for preparing assays, methods for depositing the reagents for the assays, discs for performing assays, and detection systems.

A wide variety of current diagnostic and other biochemical tests employ a substance (chromagen) that undergoes a detectable color development or change of fluorescent emission in the presence of the analyte of interest. The intensity of the color or fluorescence developed is time dependent and proportional to the concentration of the analyte of interest. For colorimetric assays, the intensity of the color is measured by optical density measurement at specific wavelengths using a spectrophotometer.

The present invention includes methods for quantifying the concentration of an analyte of interest in a biological sample on optical biodiscs using colorimetric assays. Analytes may include, for example, glucose, cholesterol, and triglycerides. In one embodiment, reagents are immobilized on the optical disc prior to the assay. To perform the assay, the sample (preferably serum, but other types of body fluids could also be used) is loaded into the channel via the injection port. After injection, the ports may be sealed, such as with tape or other suitable means. Depending on the assay protocol, the bio-disc is incubated at room temperature, or other desired temperature, for an appropriate time, e.g., 3 to 7 minutes. The optical disc reader then quantifies the intensity of the color developed. After data collection and processing, the results of the assay are displayed on a computer monitor. It should be noted that some diagnostic colorimetric assays in clinical laboratories are carried out at 37 degrees Celsius to facilitate and accelerate color development. For ease of operation, colorimetric assays performed on optical discs may advantageously be optimized to run at ambient temperature. The optimization may include selection of enzyme sources, enzymes concentrations, and sample preparation.

In one embodiment, Chromagen selection is important in optimizing colorimetric assays for optical density measurements on bio-discs since chromagens are detected at specific wavelengths. CD-R type disc readers, for example, are capable of detecting chromagens in the infrared region (750 nm to 800 nm). Other types of optical disc systems may be used in the present invention including DVD, DVD-R, fluorescent, phosphorescent, and any other similar optical disc reader. The amplitude of optical density measurements depends on the optical pathlength, the molar extinction coefficient of the chromagen and the concentration of the analyte of interest (Beer's law). To optimize the sensitivity of colorimetric assays on optical discs, several chromagens with high molar extinction coefficients at the wavelengths of interest have been identified and evaluated.

Chromagens suitable for colorimetric assays on CD-R type optical discs include, but are not limited to, N,N′-Bis(2-hydroxy-3-sulfopropyl)tolidine, disodium salt (SAT-3), N-(Carboxymethylaminocarbonyl)-4,4′-bis(dimethylamino)-diphenylamine sodium salt (DA-64), 2,2′-azino-dimethylthiozoline-6-sulfonate (ABTS), Trinder's reagents N-Ethyl-N-(2-hydroxy-3-sulfopropyl)3-methylaniline, sodium salt, dihydrate (TOOS) with the coupling reagent 3-(N-Methyl-N-phenylamino)-6-aminobenzenesulfonic acid, and sodium salt (NCP-11).

### BRIEF DESCRIPTION OF THE DRAWING FIGURES

Further objects of the present invention together with additional features contributing thereto and advantages accruing therefrom will be apparent from the following description of the preferred embodiments of the invention which are shown in the accompanying drawing figures with like reference numerals indicating like components throughout, wherein:

FIG. 1 is a pictorial representation of a bio-disc system;

FIG. 2 is an exploded perspective view of a reflective bio-disc;

FIG. 3 is a top plan view of the disc shown in FIG. 2;

FIG. 4 is a perspective view of the disc illustrated in FIG. 2 with cut-away sections showing the different layers of the disc;

FIG. 5 is an exploded perspective view of a transmissive bio-disc;

FIG. 6 is a perspective view representing the disc shown in FIG. 5 with a cut-away section illustrating the functional aspects of a semi-reflective layer of the disc;

FIG. 7 is a graphical representation showing the relationship between thickness and transmission of a thin gold film;

FIG. 8 is a top plan view of the disc shown in FIG. 5;

FIG. 9 is a perspective view of the disc illustrated in FIG. 5 with cut-away sections showing the different layers of the disc including the type of semi-reflective layer shown in FIG. 6;

FIG. 10 is a perspective and block diagram representation illustrating the system of FIG. 1 in more detail;

FIG. 11 is a partial cross sectional view taken perpendicular to a radius of the reflective optical bio-disc illustrated in FIGS. 2, 3, and 4 showing a flow channel formed therein;

FIG. 12 is a partial cross sectional view taken perpendicular to a radius of the transmissive optical bio-disc illustrated in FIGS. 5, 8, and 9 showing a flow channel formed therein and a top detector;

FIG. 13 is a partial longitudinal cross sectional view of the reflective optical bio-disc shown in FIGS. 2, 3, and 4 illustrating a wobble groove formed therein;

FIG. 14 is a partial longitudinal cross sectional view of the transmissive optical bio-disc illustrated in FIGS. 5, 8, and 9 showing a wobble groove formed therein and a top detector;

FIG. 15 is a view similar to FIG. 11 showing the entire thickness of the reflective disc and the initial refractive property thereof;

FIG. 16 is a view similar to FIG. 12 showing the entire thickness of the transmissive disc and the initial refractive property thereof;

FIG. 17A is an exploded perspective view of a reflective bio-disc incorporating equi-radial channels of the present invention;

FIG. 17B is a top plan view of the disc shown in FIG. 17A;

FIG. 17C is a perspective view of the disc illustrated in FIG. 17A with cut-away sections showing the different layers of the equi-radial reflective disc;

FIG. 18A is an exploded perspective view of a transmissive bio-disc utilizing the e-radial channels of the present invention;

FIG. 18B is a top plan view of the disc shown in FIG. 18A;

FIG. 18C is a perspective view of the disc illustrated in FIG. 18A with cut-away sections showing the different layers of this embodiment of the equi-radial transmissive bio-disc;

FIG. 19 is a graphical representation of the generation of a calibration curve for a glucose assay; and

FIG. 20 is a graphical representation of the generation of a calibration curve for a cholesterol assay.

### DETAILED DESCRIPTION OF THE INVENTION

The present invention relates in general to preparation of biomedical samples and analysis of same using an optical bio-disc system. More specifically, this invention is directed to colorimetric and fluorescent assays. The invention includes methods for preparing assays, methods for depositing the reagents for the assays, discs for performing assays, and detection systems. Each of the aspects of the present invention is discussed below in further detail.

Drive System and Related Discs

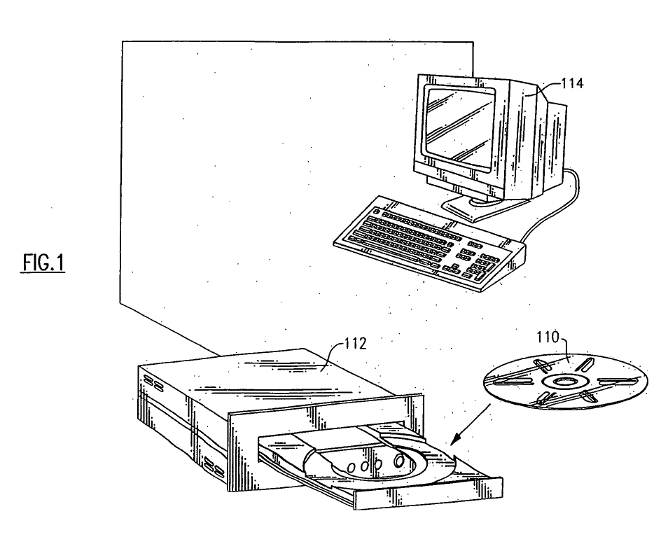

FIG. 1 is a perspective view of an optical bio-disc 110 according to the present invention as implemented to conduct the cell counts and differential cell counts disclosed herein. The present optical bio-disc 110 is shown in conjunction with an optical disc drive 112 and a display monitor 114. Further details relating to this type of disc drive and disc analysis system are disclosed in commonly assigned and co-pending U.S. patent application Ser. No. 10/008,156 entitled “Disc Drive System and Methods for Use with Bio-discs” filed Nov. 9, 2001 and U.S. patent application Ser. No. 10/043,688 entitled “Optical Disc Analysis System Including Related Methods For Biological and Medical Imaging” filed Jan. 10, 2002, both of which are herein incorporated by reference.

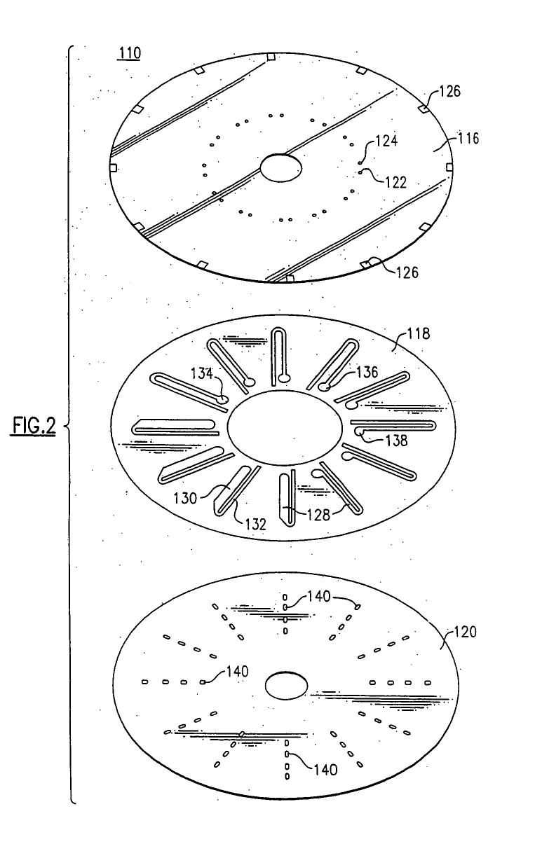

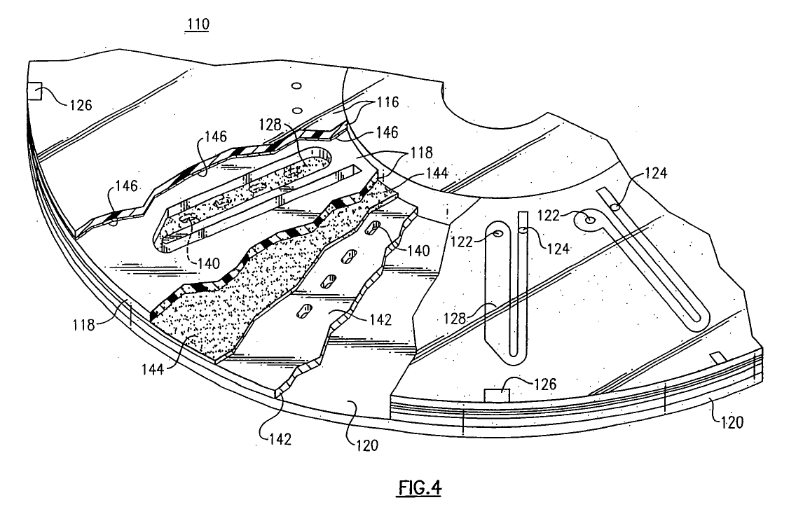

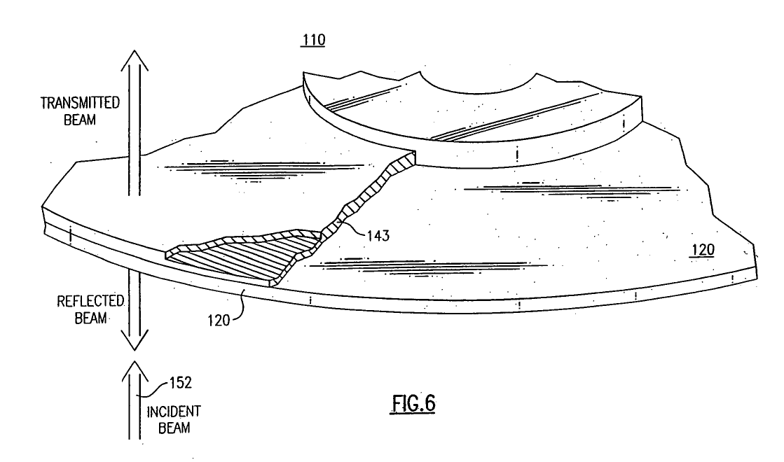

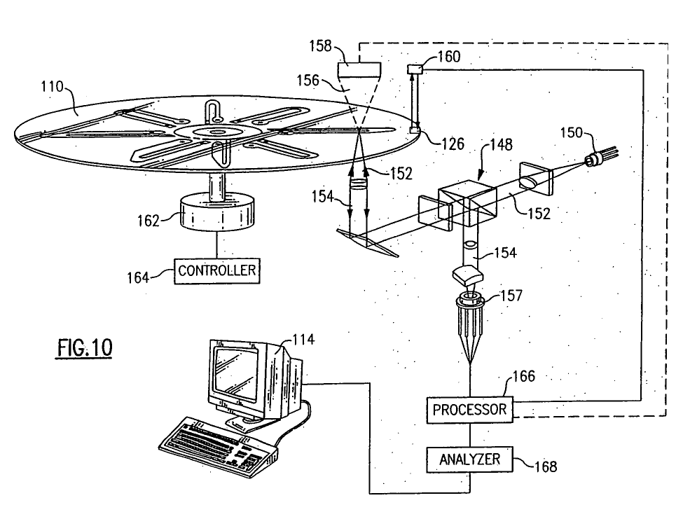

FIG. 2 is an exploded perspective view of the principal structural elements of one embodiment of the optical bio-disc 110. FIG. 2 is an example of a reflective zone optical bio-disc 110 (hereinafter “reflective disc”) that may be used in the present invention. The principal structural elements include a cap portion 116, an adhesive member or channel layer 118, and a substrate 120. The cap portion 116 includes one or more inlet ports 122 and one or more vent ports 124. The cap portion 116 may be formed from polycarbonate and is preferably coated with a reflective surface 146 (FIG. 4) on the bottom thereof as viewed from the perspective of FIG. 2. In the preferred embodiment, trigger marks or markings 126 are included on the surface of the reflective layer 142 (FIG. 4). Trigger markings 126 may include a clear window in multiple, or all, layers of the bio-disc, an opaque area, or a reflective or semi-reflective area encoded with information that sends data to a processor 166, as shown FIG. 10, that in turn interacts with the operative functions of the interrogation or incident beam 152, FIGS. 6 and 10.

The second element shown in FIG. 2 is an adhesive member or channel layer 118 having fluidic circuits 128 or U-channels formed therein. The fluidic circuits 128 are formed by stamping or cutting the membrane to remove plastic film and form the shapes as indicated. Each of the fluidic circuits 128 includes a flow channel 130 and a return channel 132. Some of the fluidic circuits 128 illustrated in FIG. 2 include a mixing chamber 134. Two different types of mixing chambers 134 are illustrated. The first is a symmetric mixing chamber 136 that is symmetrically formed relative to the flow channel 130. The second is an off-set mixing chamber 138. The off-set mixing chamber 138 is formed to one side of the flow channel 130 as indicated.

The third element illustrated in FIG. 2 is a substrate 120 including target or capture zones 140. The substrate 120 is preferably made of polycarbonate and has a reflective layer 142 deposited on the top thereof, FIG. 4. The target zones 140 are formed by removing the reflective layer 142 in the indicated shape or alternatively in any desired shape. Alternatively, the target zone 140 may be formed by a masking technique that includes masking the target zone 140 area before applying the reflective layer 142. The reflective layer 142 may be formed from a metal such as aluminum or gold.

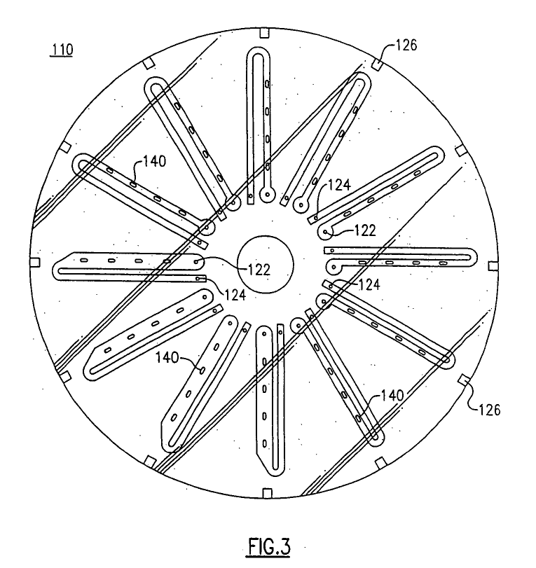

FIG. 3 is a top plan view of the optical bio-disc 110 illustrated in FIG. 2 with the reflective layer 142 on the cap portion 116 shown as transparent to reveal the fluidic circuits 128, the target zones 140, and trigger markings 126 situated within the disc.

FIG. 4 is an enlarged perspective view of the reflective zone type optical bio-disc 110 according to one embodiment of the present invention. This view includes a portion of the various layers thereof, cut away to illustrate a partial sectional view of each principal layer, substrate, coating, or membrane. FIG. 4 shows the substrate 120 that is coated with the reflective layer 142. An active layer 144 is applied over the reflective layer 142. In the preferred embodiment, the active layer 144 may be formed from polystyrene. Alternatively, polycarbonate, gold, activated glass, modified glass, or modified polystyrene, for example, polystyrene-co-maleic anhydride, may be used. In addition, hydrogels can be used. Alternatively as illustrated in this embodiment, the plastic adhesive member 118 is applied over the active layer 144. The exposed section of the plastic adhesive member 118 illustrates the cut out or stamped U-shaped form that creates the fluidic circuits 128. The final principal structural layer in this reflective zone embodiment of the present bio-disc is the cap portion 116. The cap portion 116 includes the reflective surface 146 on the bottom thereof. The reflective surface 146 may be made from a metal such as aluminum or gold.

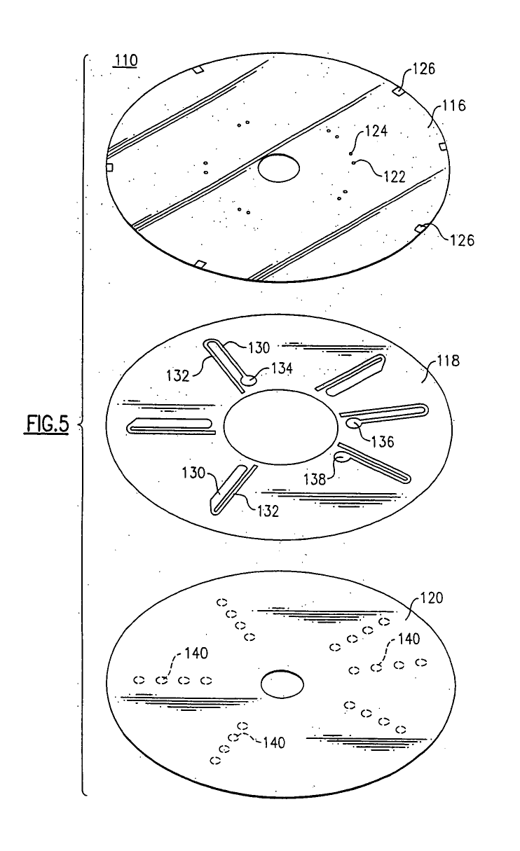

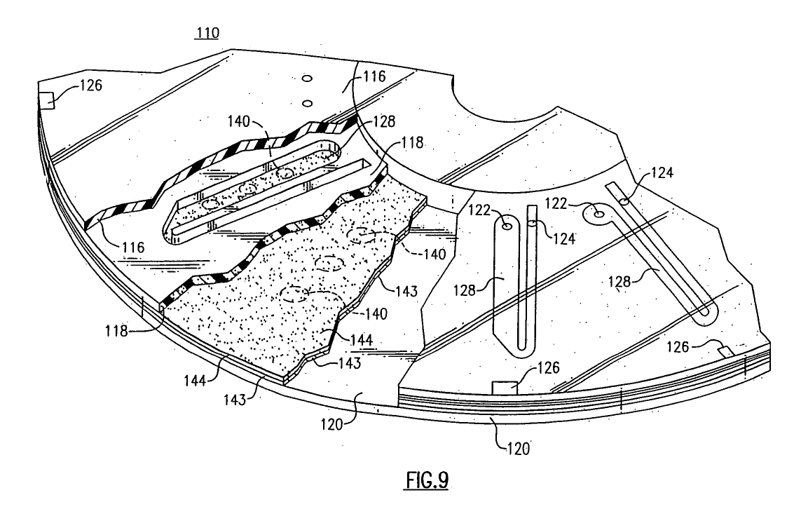

Referring now to FIG. 5, there is shown an exploded perspective view of the principal structural elements of a transmissive type of optical bio-disc 110 according to the present invention. The principal structural elements of the transmissive type of optical bio-disc 110 similarly include the cap portion 116, the adhesive or channel member 118, and the substrate 120 layer. The cap portion 116 includes one or more inlet ports 122 and one or more vent ports 124. The cap portion 116 may be formed from a polycarbonate layer. Optional trigger markings 126 may be included on the surface of a thin semi-reflective layer 143, as illustrated in FIGS. 6 and 9. Trigger markings 126 may include a clear window in all three layers of the bio-disc, an opaque area, or a reflective or semi-reflective area encoded with information that sends data to the processor 166, FIG. 10, which in turn interacts with the operative functions of the interrogation beam 152, FIGS. 6 and 10.

The second element shown in FIG. 5 is the adhesive member or channel layer 118 having fluidic circuits 128 or U-channels formed therein. The fluidic circuits 128 are formed by stamping or cutting the membrane to remove plastic film and form the shapes as indicated. Each of the fluidic circuits 128 includes the flow channel 130 and the return channel 132. Some of the fluidic circuits 128 illustrated in FIG. 5 include the mixing chamber 134. Two different types of mixing chambers 134 are illustrated. The first is the symmetric mixing chamber 136 that is symmetrically formed relative to the flow channel 130. The second is the off-set mixing chamber 138. The off-set mixing chamber 138 is formed to one side of the flow channel 130 as indicated.

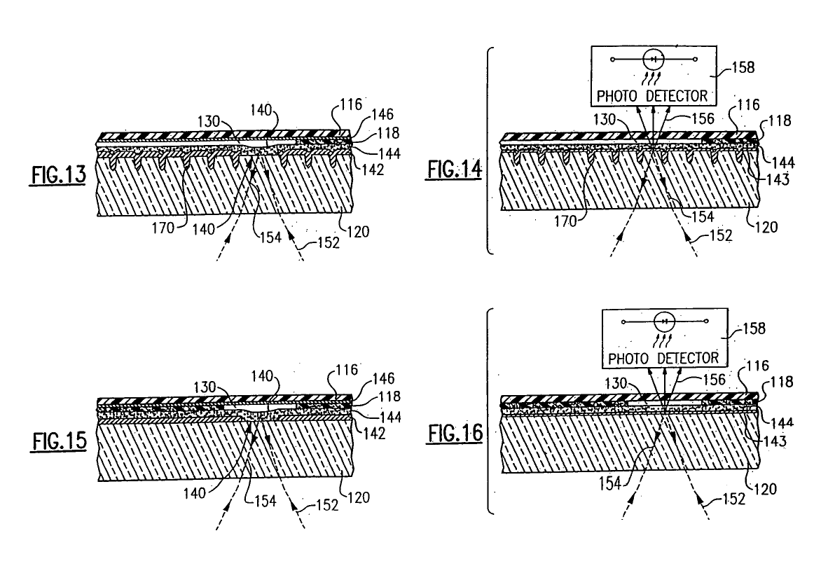

The third element illustrated in FIG. 5 is the substrate 120, which may include the target or capture zones 140. The substrate 120 is preferably made of polycarbonate and has the thin semi-reflective layer 143 deposited on the top thereof, FIG. 6. The semi-reflective layer 143 associated with the substrate 120 of the disc 110 illustrated in FIGS. 5 and 6 may be significantly thinner than the reflective layer 142 on the substrate 120 of the reflective disc 110 illustrated in FIGS. 2, 3 and 4. The thinner semi-reflective layer 143 allows for some transmission of the interrogation beam 152 through the structural layers of the transmissive disc as shown in FIGS. 6 and 12. The thin semi-reflective layer 143 may be formed from a metal such as aluminum or gold.

FIG. 6 is an enlarged perspective view of the substrate 120 and semi-reflective layer 143 of the transmissive embodiment of the optical bio-disc 110 illustrated in FIG. 5. The thin semi-reflective layer 143 may be made from a metal such as aluminum or gold. In the preferred embodiment, the thin semi-reflective layer 143 of the transmissive disc illustrated in FIGS. 5 and 6 is approximately 100-300 Å thick and does not exceed 400 Å. This thinner semi-reflective layer 143 allows a portion of the incident or interrogation beam 152 to penetrate and pass through the semi-reflective layer 143 to be detected by a top detector 158, FIGS. 10 and 12, while some of the light is reflected or returned back along the incident path. As indicated below, Table 1 presents the reflective and transmissive characteristics of a gold film relative to the thickness of the film. The gold film layer is fully reflective at a thickness greater than 800 Å, while the threshold density for transmission of light through the gold film is approximately 400 Å.

TABLE 1
Au Film Reflection and Transmission (Absolute Values)

| Thickness (Angstroms) | Thickness (nm) | Reflectance | Transmittance |
|---|---|---|---|
| 0 | 0 | 0.0505 | 0.9495 |
| 50 | 5 | 0.1683 | 0.7709 |
| 100 | 10 | 0.3981 | 0.5169 |
| 150 | 15 | 0.5873 | 0.3264 |
| 200 | 20 | 0.7142 | 0.2057 |
| 250 | 25 | 0.7959 | 0.1314 |
| 300 | 30 | 0.8488 | 0.0851 |
| 350 | 35 | 0.8836 | 0.0557 |
| 400 | 40 | 0.9067 | 0.0368 |
| 450 | 45 | 0.9222 | 0.0244 |
| 500 | 50 | 0.9328 | 0.0163 |
| 550 | 55 | 0.9399 | 0.0109 |
| 600 | 60 | 0.9448 | 0.0073 |
| 650 | 65 | 0.9482 | 0.0049 |
| 700 | 70 | 0.9505 | 0.0033 |
| 750 | 75 | 0.9520 | 0.0022 |
| 800 | 80 | 0.9531 | 0.0015 |

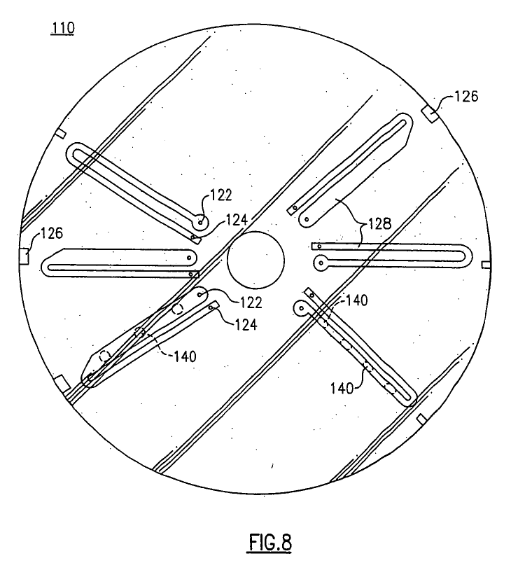

With reference next to FIG. 8, there is shown a top plan view of the transmissive type optical bio-disc 110 illustrated in FIGS. 5 and 6 with the transparent cap portion 116 revealing the fluidic channels, the trigger markings 126, and the target zones 140 as situated within the disc.

FIG. 9 is an enlarged perspective view of the optical bio-disc 110 according to the transmissive disc embodiment of the present invention. The disc 110 is illustrated with a portion of the various layers thereof cut away to show a partial sectional view of each principal layer, substrate, coating, or membrane. FIG. 9 illustrates a transmissive disc format with the clear cap portion 116, the thin semi-reflective layer 143 on the substrate 120, and trigger markings 126. In this embodiment, trigger markings 126 include opaque material placed on the top portion of the cap. Alternatively the trigger marking 126 may be formed by clear, non-reflective windows etched on the thin reflective layer 143 of the disc, or any mark that absorbs or does not reflect the signal coming from the trigger detector 160, FIG. 10. FIG. 9 also shows the target zones 140 formed by marking the designated area in the indicated shape or alternatively in any desired shape. Markings to indicate target zone 140 may be made on the thin semi-reflective layer 143 on the substrate 120 or on the bottom portion of the substrate 120 (under the disc). Alternatively, the target zones 140 may be formed by a masking technique that includes masking all, or a portion, of the thin semi-reflective layer 143 except the target zones 140. In this embodiment, target zones 140 may be created by silk screening ink onto the thin semi-reflective layer 143. In the transmissive disc format illustrated in FIGS. 5, 8, and 9, the target zones 140 may alternatively be defined by address information encoded on the disc. In this embodiment, target zones 140 do not include a physically discernable edge boundary.

With continuing reference to FIG. 9, an active layer 144 is illustrated as applied over the thin semi-reflective layer 143. In the preferred embodiment, the active layer 144 is a 10 to 200 μm thick layer of 2% polystyrene. Alternatively, polycarbonate, gold, activated glass, modified glass, or modified polystyrene, for example, polystyrene-co-maleic anhydride, may be used. In addition, hydrogels can be used. As illustrated in this embodiment, the plastic adhesive member 118 is applied over the active layer 144. The exposed section of the plastic adhesive member 118 illustrates the cut out or stamped U-shaped form that creates the fluidic circuits 128.

The final principal structural layer in this transmissive embodiment of the present bio-disc 110 is the clear, non-reflective cap portion 116 that includes inlet ports 122 and vent ports 124.

Referring now to FIG. 10, there is a representation in perspective and block diagram illustrating optical components 148, a light source 150 that produces the incident or interrogation beam 152, a return beam 154, and a transmitted beam 156. In the case of the reflective bio-disc illustrated in FIG. 4, the return beam 154 is reflected from the reflective surface 146 of the cap portion 116 of the optical bio-disc 110. In this reflective embodiment of the present optical bio-disc 110, the return beam 154 is detected and analyzed for the presence of signal elements by a bottom detector 157. In the transmissive bio-disc format, on the other hand, the transmitted beam 156 is detected, by a top detector 158, and is also analyzed for the presence of signal elements. In the transmissive embodiment, a photo detector may be used as a top detector 158.

FIG. 10 also shows a hardware trigger mechanism that includes the trigger markings 126 on the disc and a trigger detector 160. The hardware triggering mechanism is used in both reflective bio-discs (FIG. 4) and transmissive bio-discs (FIG. 9). The triggering mechanism allows the processor 166 to collect data when the interrogation beam 152 is on a respective target zone 140. Furthermore, in the transmissive bio-disc system, a software trigger may also be used. The software trigger uses the bottom detector to signal the processor 166 to collect data as soon as the interrogation beam 152 hits the edge of a respective target zone 140. FIG. 10 further illustrates a drive motor 162 and a controller 164 for controlling the rotation of the optical bio-disc 110. FIG. 10 also shows the processor 166 and analyzer 168 implemented in the alternative for processing the return beam 154 and transmitted beam 156 associated the transmissive optical bio-disc.

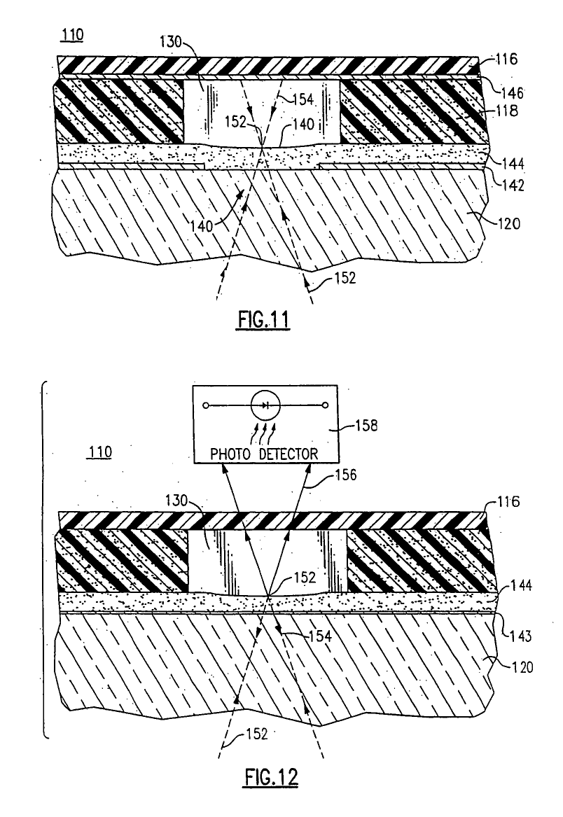

As shown in FIG. 11, there is presented a partial cross sectional view of the reflective disc embodiment of the optical bio-disc 110 according to the present invention. FIG. 11 illustrates the substrate 120 and the reflective layer 142. As indicated above, the reflective layer 142 may be made from a material such as aluminum, gold or other suitable reflective material. In this embodiment, the top surface of the substrate 120 is smooth. FIG. 11 also shows the active layer 144 applied over the reflective layer 142. As also shown in FIG. 11, the target zone 140 is formed by removing an area or portion of the reflective layer 142 at a desired location or, alternatively, by masking the desired area prior to applying the reflective layer 142. As further illustrated in FIG. 11, the plastic adhesive member 118 is applied over the active layer 144. FIG. 11 also shows the cap portion 116 and the reflective surface 146 associated therewith. Thus when the cap portion 116 is applied to the plastic adhesive member 118 including the desired cutout shapes, flow channel 130 is thereby formed. As indicated by the arrowheads shown in FIG. 11, the path of the incident beam 152 is initially directed toward the substrate 120 from below the disc 110. The incident beam then focuses at a point proximate the reflective layer 142. Since this focusing takes place in the target zone 140 where a portion of the reflective layer 142 is absent, the incident light continues along a path through the active layer 144 and into the flow channel 130. The incident beam 152 then continues upwardly traversing through the flow channel to eventually fall incident onto the reflective surface 146. At this point, the incident beam 152 is returned or reflected back along the incident path and thereby forms the return beam 154.

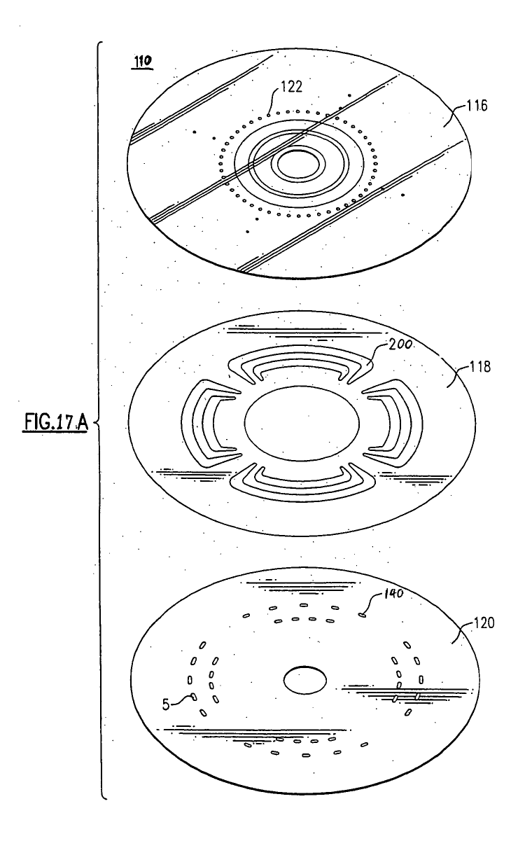

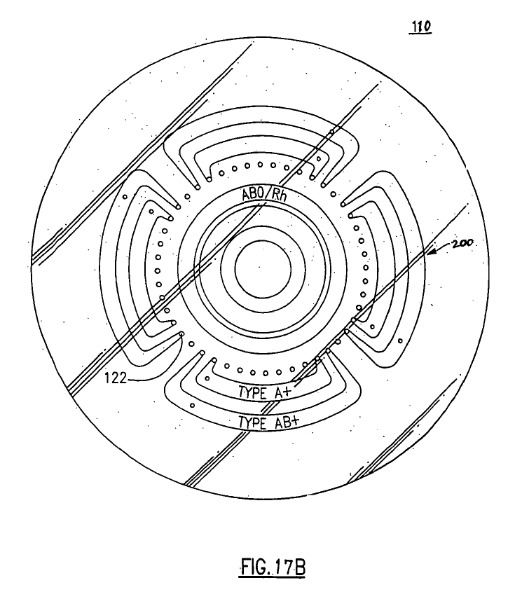

FIG. 12 is a partial cross sectional view of the transmissive embodiment of the bio-disc 110 according to the present invention. FIG. 12 illustrates a transmissive disc format with the clear cap portion 116 and the thin semi-reflective layer 143 on the substrate 120. FIG. 12 also shows the active layer 144 applied over the thin semi-reflective layer 143. In the preferred embodiment, the transmissive disc has the thin semi-reflective layer 143 made from a metal such as aluminum or gold approximately 100-300 Angstroms thick and does not exceed 400 Angstroms. This thin semi-reflective layer 143 allows a portion of the incident or interrogation beam 152, from the light source 150, FIG. 10, to penetrate and pass upwardly through the disc to be detected by a top detector 158, while some of the light is reflected back along the same path as the incident beam but in the opposite direction. In this arrangement, the return or reflected beam 154 is reflected from the semi-reflective layer 143. Thus in this manner, the return beam 154 does not enter into the flow channel 130. The reflected light or return beam 154 may be used for tracking the incident beam 152 on pre-recorded information tracks formed in or on the semi-reflective layer 143 as described in more detail in conjunction with FIGS. 13 and 14. In the disc embodiment illustrated in FIG. 12, a physically defined target zone 140 may or may not be present. Target zone 140 may be created by direct markings made on the thin semi-reflective layer 143 on the substrate 120. These marking may be formed using silk screening or any equivalent method. In the alternative embodiment where no physical indicia are employed to define a target zone (such as, for example, when encoded software addressing is utilized) the flow channel 130 in effect may be employed as a confined target area in which inspection of an investigational feature is conducted.

FIG. 13 is a cross sectional view taken across the tracks of the reflective disc embodiment of the bio-disc 110 according to the present invention. This view is taken longitudinally along a radius and flow channel of the disc. FIG. 13 includes the substrate 120 and the reflective layer 142. In this embodiment, the substrate 120 includes a series of grooves 170. The grooves 170 are in the form of a spiral extending from near the center of the disc toward the outer edge. The grooves 170 are implemented so that the interrogation beam 152 may track along the spiral grooves 170 on the disc. This type of groove 170 is known as a “wobble groove”. A bottom portion having undulating or wavy sidewalls forms the groove 170, while a raised or elevated portion separates adjacent grooves 170 in the spiral. The reflective layer 142 applied over the grooves 170 in this embodiment is, as illustrated, conformal in nature. FIG. 13 also shows the active layer 144 applied over the reflective layer 142. As shown in FIG. 13, the target zone 140 is formed by removing an area or portion of the reflective layer 142 at a desired location or, alternatively, by masking the desired area prior to applying the reflective layer 142. As further illustrated in FIG. 13, the plastic adhesive member 118 is applied over the active layer 144. FIG. 13 also shows the cap portion 116 and the reflective surface 146 associated therewith. Thus, when the cap portion 116 is applied to the plastic adhesive member 118 including the desired cutout shapes, the flow channel 130 is thereby formed.

FIG. 14 is a cross sectional view taken across the tracks of the transmissive disc embodiment of the bio-disc 110 according to the present invention as described in FIG. 12, for example. This view is taken longitudinally along a radius and flow channel of the disc. FIG. 14 illustrates the substrate 120 and the thin semi-reflective layer 143. This thin semi-reflective layer 143 allows the incident or interrogation beam 152, from the light source 150, to penetrate and pass through the disc to be detected by the top detector 158, while some of the light is reflected back in the form of the return beam 154. The thickness of the thin semi-reflective layer 143 is determined by the minimum amount of reflected light needed by the disc reader to maintain its tracking ability. The substrate 120 in this embodiment, like that discussed in FIG. 13, includes the series of grooves 170. The grooves 170 in this embodiment are also preferably in the form of a spiral extending from near the center of the disc toward the outer edge. The grooves 170 are implemented so that the interrogation beam 152 may track along the spiral. FIG. 14 also shows the active layer 144 applied over the thin semi-reflective layer 143. As further illustrated in FIG. 14, the plastic adhesive member or channel layer 118 is applied over the active layer 144. FIG. 14 also shows the cap portion 116 without a reflective surface 146. Thus, when the cap is applied to the plastic adhesive member 118 including the desired cutout shapes, the flow channel 130 is thereby formed and a part of the incident beam 152 is allowed to pass therethrough substantially unreflected.

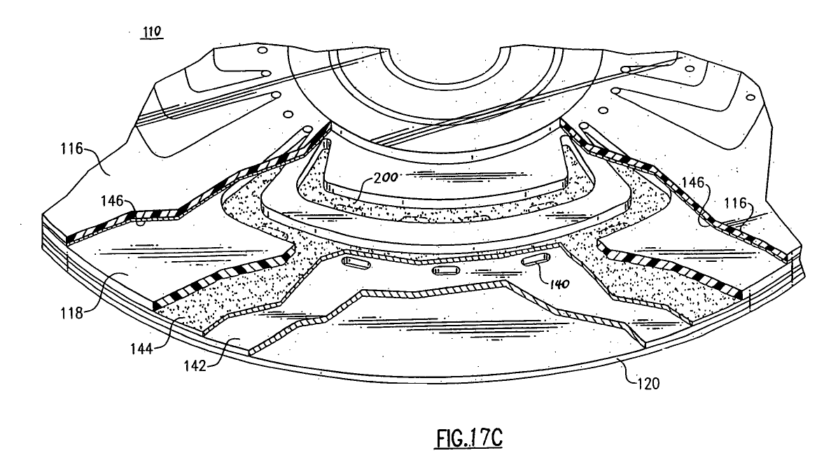

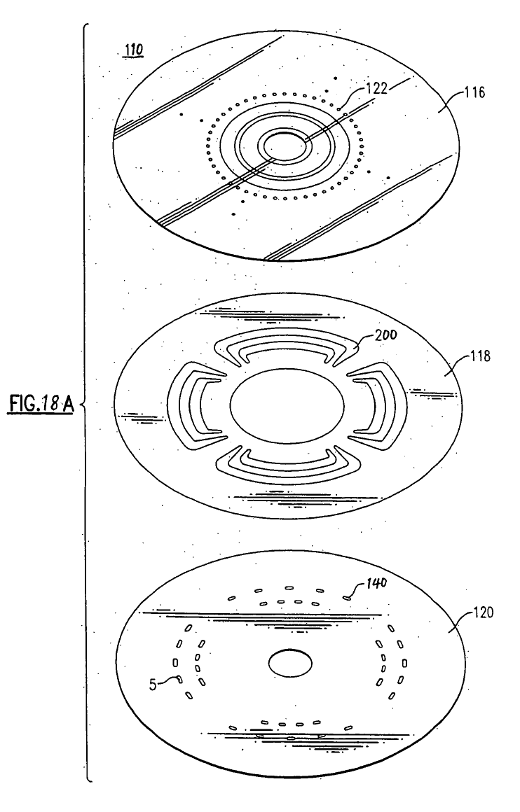

FIG. 15 is a view similar to FIG. 11 showing the entire thickness of the reflective disc and the initial refractive property thereof. FIG. 16 is a view similar to FIG. 12 showing the entire thickness of the transmissive disc and the initial refractive property thereof. Grooves 170 are not seen in FIGS. 15 and 16 since the sections are cut along the grooves 170. FIGS. 15 and 16 show the presence of the narrow flow channel 130 that is situated perpendicular to the grooves 170 in these embodiments. FIGS. 13, 14, 15, and 16 show the entire thickness of the respective reflective and transmissive discs. In these figures, the incident beam 152 is illustrated initially interacting with the substrate 120 which has refractive properties that change the path of the incident beam as illustrated to provide focusing of the beam 152 on the reflective layer 142 or the thin semi-reflective layer 143.

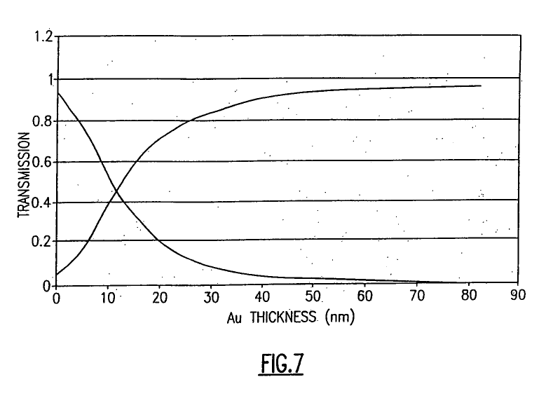

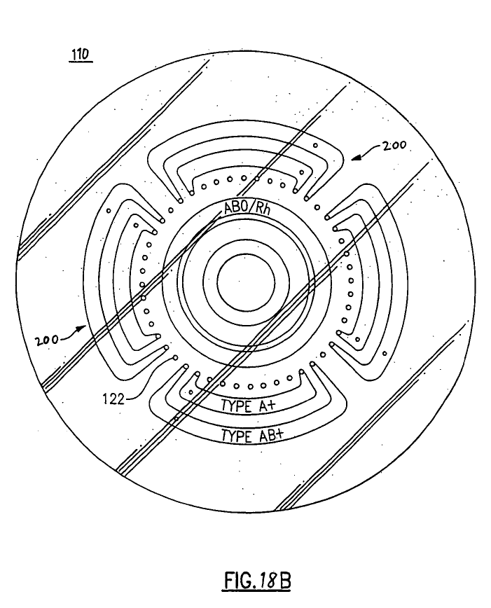

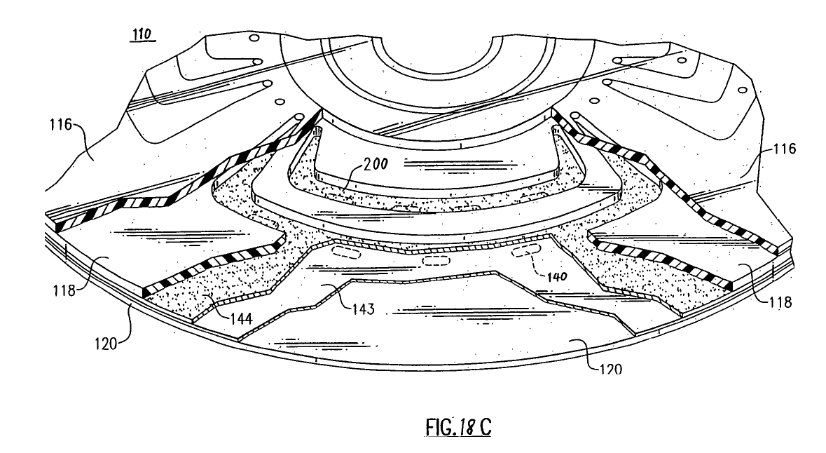

Alternative embodiments of the bio-disc according to the present invention will now be described with reference to FIGS. 17A, 17B, 17C, 18A, 18B, and 18C. Various features of the discs of these latter embodiments have been already illustrated with reference to FIGS. 1 to 16, and therefore such common features will not be described again in the following. Accordingly, and for the sake of simplicity, as a general rule in FIGS. 17 and 18, the features differentiating the bio-disc 110 from those of FIGS. 1 to 21 are represented.

Furthermore, the following description of the bio-disc of the invention can be readily applied to a transmissive-type as well as to a reflective-type optical bio-disc described above in conjunction with FIGS. 2 to 9.

FIG. 17A is an exploded perspective view of a reflective bio-disc incorporating equi-radial channels 200 of the present invention. This general construction corresponds to the radial-channel disc shown in FIG. 2. The e-rad or eRad implementation of the bio-disc 110 shown in FIG. 17A similarly includes the cap 116, the channel layer 118, and the substrate 120. The channel layer 118 includes the equi-radial fluid channels 200, while the substrate 120 includes the corresponding arrays of target zones 140.

FIG. 17B is a top plan view of the disc shown in FIG. 17A. FIG. 17B further shows a top plan view of an embodiment of eRad disc with a transparent cap portion, which disc has two tiers of circumferential fluid channels with ABO chemistry and two blood types (A+ and AB+). As shown in FIG. 17B, it is also possible to provide a priori, at the manufacturing stage of the disc of the invention, a plurality of entry ports, eventually at different radial coordinate, so that a range of equi-radial, spiralling, or radial reaction sites and/or channels are possible on one disc. These channels can be used for different test suites, or for multiple samples of single test suites.

FIG. 17C is a perspective view of the disc illustrated in FIG. 17A with cut-away sections showing the different layers of the e-radial reflective disc. This view is similar to the reflective disc shown in FIG. 4. The e-rad implementation of the reflective bio-disc shown in FIG. 17C similarly includes the reflective layer 142, active layer 144 as applied over the reflective layer 142, and the reflective layer 146 on the cap portion 116.

FIG. 18A is an exploded perspective view of a transmissive bio-disc utilizing the e-radial channels of the present invention. This general construction corresponds to the radial-channel disc shown in FIG. 5. The transmissive e-rad implementation of the bio-disc 110 shown in FIG. 18A similarly includes the cap 116, the channel layer 118, and the substrate 120. The channel layer 118 includes the equi-radial fluid channels 200, while the substrate 120 includes the corresponding arrays of target zones 140.

FIG. 18B is a top plan view of the transmissive e-rad disc shown in FIG. 18A. FIG. 18B further shows two tiers of circumferential fluid channels with ABO chemistry and two blood types (A+ and AB+). As previously discussed, the assays are performed in the target, capture, or analysis zones 140.

FIG. 18C is a perspective view of the disc illustrated in FIG. 18A with cut-away sections showing the different layers of this embodiment of the e-rad transmissive bio-disc. This view is similar to the transmissive disc shown in FIG. 9. The e-rad implementation of the transmissive bio-disc shown in FIG. 18C similarly includes the thin semi-reflective layer 143 and the active layer 144 as applied over the thin semi-reflective layer 143.

Quantification of Glucose and Cholesterol Using the Optical Bio-Disc

A criterion that defines a good diagnostic assay is the ease by which one performs the assay. For colorimetric assays on optical bio-discs, the reagents used for the assay may advantageously be immobilized on the disc prior to the assay. There are several methods that can be used for reagent deposition. They include air or vacuum evaporation, enzyme immobilization by chemical linkage, lyophilization, or reagent printing on a suitable medium (i.e. filter paper or membrane strips). The above methods have been tested on bio-discs. In an advantageous embodiment, a reagent printing process is used to apply the reagents on the membrane strips because reagent stability for several weeks or months is preserved. In one embodiment, the printing process may be performed using a printing device, such as an ink jet printer.

For each assay, the reagents are printed on 3×5×0.3 mm strips. The printing can be done manually with a pipettor, or by automatic applicators. The volume of reagents deposited on the strips varies from 2 to 5 ul. The strips are deposited on the bio-disc at the time of assembly. The thickness of the reagent strips is such that they will fit securely within the channels of the bio-disc.

The selection of membrane strips for reagent deposition affects the success of the assay. Membrane strips are traditionally used in dipstick or lateral flow assays, where the chemistry typically occurs on a solid phase. However, for colorimetric assays on optical analysis discs, the chemistry between the sample and the reagents occurs in solution. For this reason, the use of membrane strips in colorimetric assays on bio-discs is rather unique. Further, instead of using nitrocellulose membranes that are normally used in lateral flow assays, the membrane strips chosen for reagent deposition in colorimetric assays should have a good absorbing capacity to accommodate the volume of reagent deposited, while retaining good release efficiency. A membrane strip with good release efficiency allows the reagents to be released from the storage medium (membrane strip) into solution as soon as the sample is injected into the reaction chamber, where they effectively catalyze the desired reactions. This allows for the color development from the reaction to be homogenous throughout the reaction chamber. The membrane strips for reagent deposition can be prepared independently of the discs and easily deposited within the disc during disc assembly. Numerous membrane strips have been tested for this particular function. In one embodiment, a membrane strip for reagent deposition is a hydrophilic polyethersulfone membrane of pore size 0.2 um or above (Pall, Port Washington, New York). In another embodiment, a membrane strip for reagent deposition is a bibulous hydrophilic material. Those of skill in the art will also recognized that other materials that have the above discussed properties may readily be used for membrane strips.

On optical bio-discs, calibrators that are normally used in colorimetric assays may be replaced by calibration bars, which express the concentrations of the calibrators in terms of the relative amount of light transmitted or reflected. The calibration bars could be created either in the software or directly on the disc. The creation of calibration bars reduces the assay time significantly and makes the assay much more user friendly.

According to one aspect of the present invention, there are provided detection methods for quantifying the concentration of an analyte of interest in a biological sample on the bio-discs. The detection includes directing a beam of electromagnetic energy from a disc drive toward the capture field and analyzing electromagnetic energy returned from or transmitted through the capture field.

The optical density change in colorimetric assays can be quantified by the optical disc reader by two related ways. These include measuring the change in light either reflected or transmitted. The disc may be referred to as reflective, transmissive, or some combination of reflective and transmissive. In a reflective disc, an incident light beam is focused onto the disc (typically at a reflective surface where information is encoded), reflected, and returned through optical elements to a detector on the same side of the disc as the light source. In a transmissive disc, light passes through the disc (or portions thereof) to a detector on the other side of the disc from the light source. In a transmissive portion of a disc, some light may also be reflected and detected as reflected light. Different detection systems are used for different types of bio-discs (top versus bottom detector).

The conversion of data captured by the CD reader into meaningful concentration units is mediated via data processing software specific for the assay of interest. In one embodiment, the data captured by the CD reader may be used to determine additional characteristics of, or related to, the assay, such as an amount of a target substance present.

The apparatus and methods in embodiments of the present invention can be designed for use by an end-user, inexpensively, without specialized expertise and expensive equipment. The system can be made portable, and thus usable in remote locations where traditional diagnostic equipment may not generally be available.

Alternatively, fluorescent assays can be carried out to quantify the concentration of an analyte of interest in a biological sample on the optical discs. In this case, the energy source in the disc drive preferably has a wavelength controllable light source and a detector that is or can be made specific to a particular wavelength. Alternatively, a disc drive can be made with a specific light source and detector to produce a dedicated device, in which case the source may need fine-tuning.

More specifically, the present invention is directed to sample preparation and generation of calibration bars for colorimetric and fluorescent assays as implemented on optical analysis discs.

A criterion that defines a good diagnostic assay is the ease by which one performs the assay. For colorimetric assays on optical bio-discs, the reagents used for the assay may be immobilized on the disc prior to the assay. At the time of the assay, the end-user just needs to dilute the sample with water then injects the sample into the channel. Alternatively, undiluted samples may be used directly.

Colorimetric assays on bio-disc can use either serum or blood as sample sources. Serum can be a direct substrate for the assays. Blood can also be used as sample source by selective filtration of red blood cells using membranes such as HemaSep or CytoSep (Pall, Port Washington, New York).

In lab-based colorimetric assays, the concentrations of unknown samples were normally derived from calibrators or solutions with known concentrations. The use of calibrators necessitated additional preparation steps, which were more time-consuming and error prone. On optical bio-discs, calibrators in colorimetric assays may be replaced by calibration bars. The creation of calibrator bars is achieved by measuring the amount of light transmitted or reflected by known concentrations of analytes. The amount of light transmitted or reflected may then be expressed relative to the minimum and maximum amount of light transmitted or reflected. The maximum amount of light transmitted or reflected may be obtained in the absence of any solution in the reaction zone. The minimum amount of light transmitted or reflected may be the amount of light transmitted or reflected from a blocked reaction zone. The blocking can be mediated with any available light blocking structure, such as a piece of black tape, for example. The calibration bars could be created either in the software or directly on the disc.

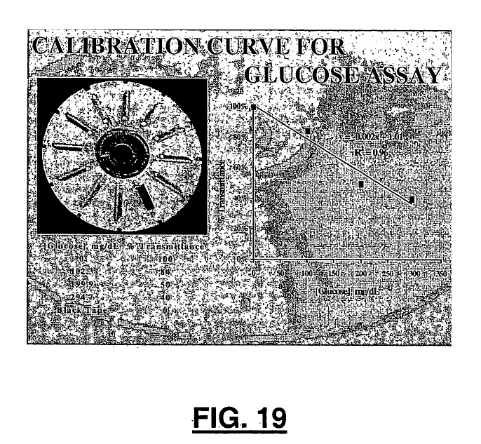

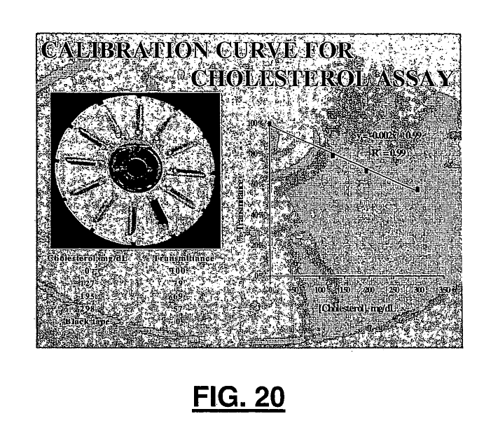

FIGS. 19 and 20 illustrate the generation of calibration curves for the glucose and cholesterol assays, respectively. The first step in the generation of the calibration curves was filling the fluidic channel or analysis chambers with calibrators of known concentrations. One analysis chamber was left empty to measure the maximum of light that can be transmitted. Another analysis chamber was blocked with a black tape; the voltage measured in that channel represents the minimum of light that can be transmitted or reflected. The table illustrated in the figures expresses the percentage of light transmitted by the calibrators with respect to the references. The calibration curves shown expresses the inverse relationship between the calibrator concentrations and the amount of light transmitted or reflected.

Other Implementations of the Current Invention

This invention or different aspects thereof may be readily implemented in or adapted to many of the discs, assays, and systems disclosed in the following commonly assigned and co-pending patent applications: U.S. patent application Ser. No. 09/378,878 entitled “Methods and Apparatus for Analyzing Operational and Non-operational Data Acquired from Optical Discs” filed Aug. 23, 1999; U.S. Provisional Patent Application Ser. No. 60/150,288 entitled “Methods and Apparatus for Optical Disc Data Acquisition Using Physical Synchronization Markers” filed Aug. 23, 1999; U.S. patent application Ser. No. 09/421,870 entitled “Trackable Optical Discs with Concurrently Readable Analyte Material” filed Oct. 26, 1999; U.S. patent application Ser. No. 09/643,106 entitled “Methods and Apparatus for Optical Disc Data Acquisition Using Physical Synchronization Markers” filed Aug. 21, 2000; U.S. patent application Ser. No. 09/999,274 entitled “Optical Biodiscs with Reflective Layers” filed Nov. 15, 2001; U.S. patent application Ser. No. 09/988,728 entitled “Methods and Apparatus for Detecting and Quantifying Lymphocytes with Optical Biodiscs” filed Nov. 16, 2001; U.S. patent application Ser. No. 09/988,850 entitled “Methods and Apparatus for Blood Typing with Optical Bio-discs” filed Nov. 19, 2001; U.S. patent application Ser. No. 09/989,684 entitled “Apparatus and Methods for Separating Agglutinants and Disperse Particles” filed Nov. 20, 2001; U.S. patent application Ser. No. 09/997,741 entitled “Dual Bead Assays Including Optical Biodiscs and Methods Relating Thereto” filed Nov. 27, 2001; U.S. patent application Ser. No. 09/997,895 entitled “Apparatus and Methods for Separating Components of Particulate Suspension” filed Nov. 30, 2001; U.S. patent application Ser. No. 10/005,313 entitled “Optical Discs for Measuring Analytes” filed Dec. 7, 2001; U.S. patent application Ser. No. 10/006,371 entitled “Methods for Detecting Analytes Using Optical Discs and Optical Disc Readers” filed Dec. 10, 2001; U.S. patent application Ser. No. 10/006,620 entitled “Multiple Data Layer Optical Discs for Detecting Analytes” filed Dec. 10, 2001; U.S. patent application Ser. No. 10/006,619 entitled “Optical Disc Assemblies for Performing Assays” filed Dec. 10, 2001; U.S. patent application Ser. No. 10/020,140 entitled “Detection System For Disk-Based Laboratory and Improved Optical Bio-Disc Including Same” filed Dec. 14, 2001; U.S. patent application Ser. No. 10/035,836 entitled “Surface Assembly for Immobilizing DNA Capture Probes and Bead-Based Assay Including Optical Bio-Discs and Methods Relating Thereto” filed Dec. 21, 2001; U.S. patent application Ser. No. 10/038,297 entitled “Dual Bead Assays Including Covalent Linkages for Improved Specificity and Related Optical Analysis Discs” filed Jan. 4, 2002; U.S. patent application Ser. No. 10/043,688 entitled “Optical Disc Analysis System Including Related Methods for Biological and Medical Imaging” filed Jan. 10, 2002; U.S. Provisional Application Ser. No. 60/348,767 entitled “Optical Disc Analysis System Including Related Signal Processing Methods and Software” filed Jan. 14, 2002 U.S. patent application Ser. No. 10/086,941 entitled “Methods for DNA Conjugation Onto Solid Phase Including Related Optical Biodiscs and Disc Drive Systems” filed Feb. 26, 2002; U.S. patent application Ser. No. 10/087,549 entitled “Methods for Decreasing Non-Specific Binding of Beads in Dual Bead Assays Including Related Optical Biodiscs and Disc Drive Systems” filed Feb. 28, 2002; U.S. patent application Ser. No. 10/099,256 entitled “Dual Bead Assays Using Cleavable Spacers and/or Ligation to Improve Specificity and Sensitivity Including Related Methods and Apparatus” filed Mar. 14, 2002; U.S. patent application Ser. No. 10/099,266 entitled “Use of Restriction Enzymes and Other Chemical Methods to Decrease Non-Specific Binding in Dual Bead Assays and Related Bio-Discs, Methods, and System Apparatus for Detecting Medical Targets” also filed Mar. 14, 2002; U.S. patent application Ser. No. 10/121,281 entitled “Multi-Parameter Assays Including Analysis Discs and Methods Relating Thereto” filed Apr. 11, 2002; U.S. patent application Ser. No. 10/150,575 entitled “Variable Sampling Control for Rendering Pixelization of Analysis Results in a Bio-Disc Assembly and Apparatus Relating Thereto” filed May 16, 2002; U.S. patent application Ser. No. 10/150,702 entitled “Surface Assembly For Immobilizing DNA Capture Probes in Genetic Assays Using Enzymatic Reactions to Generate Signals in Optical Bio-Discs and Methods Relating Thereto” filed May 16, 2002; U.S. patent application Ser. No. 10/194,418 entitled “Optical Disc System and Related Detecting and Decoding Methods for Analysis of Microscopic Structures” filed Jul. 12, 2002; U.S. patent application Ser. No. 10/194,396 entitled “Multi-Purpose Optical Analysis Disc for Conducting Assays and Various Reporting Agents for Use Therewith” also filed Jul. 12, 2002; U.S. patent application Ser. No. 10/199,973 entitled “Transmissive Optical Disc Assemblies for Performing Physical Measurements and Methods Relating Thereto” filed Jul. 19, 2002; U.S. patent application Ser. No. 10/201,591 entitled “Optical Analysis Disc and Related Drive Assembly for Performing Interactive Centrifugation” filed Jul. 22, 2002; U.S. patent application Ser. No. 10/205,011 entitled “Method and Apparatus for Bonded Fluidic Circuit for Optical Bio-Disc” filed Jul. 24, 2002; U.S. patent application Ser. No. 10/205,005 entitled “Magnetic Assisted Detection of Magnetic Beads Using Optical Disc Drives” also filed Jul. 24, 2002; U.S. patent application Ser. No. 10/230,959 entitled “Methods for Qualitative and Quantitative Analysis of Cells and Related Optical Bio-Disc Systems” filed Aug. 29, 2002; U.S. patent application Ser. No. 10/233,322 entitled “Capture Layer Assemblies for Cellular Assays Including Related Optical Analysis Discs and Methods” filed Aug. 30, 2002; U.S. patent application Ser. No. 10/236,857 entitled “Nuclear Morphology Based Identification and Quantification of White Blood Cell Types Using Optical Bio-Disc Systems” filed Sep. 6, 2002; U.S. patent application Ser. No. 10/241,512 entitled “Methods for Differential Cell Counts Including Related Apparatus and Software for Performing Same” filed Sep. 11, 2002; U.S. patent application Ser. No. 10/279,677 entitled “Segmented Area Detector for Biodrive and Methods Relating Thereto” filed Oct. 24, 2002; U.S. patent application Ser. No. 10/293,214 entitled “Optical Bio-Discs and Fluidic Circuits for Analysis of Cells and Methods Relating Thereto” filed on Nov. 13, 2002; U.S. patent application Ser. No. 10/298,263 entitled “Methods and Apparatus for Blood Typing with Optical Bio-Discs” filed on Nov. 15, 2002; U.S. patent application Ser. No. 10/307,263 entitled “Magneto-Optical Bio-Discs and Systems Including Related Methods” filed Nov. 27, 2002; U.S. patent application Ser. No. 10/341,326 entitled “Method and Apparatus for Visualizing Data” filed Jan. 13, 2003; U.S. patent application Ser. No. 10/345,122 entitled “Methods and Apparatus for Extracting Data From an Optical Analysis Disc” filed on Jan. 14, 2003; U.S. patent application Ser. No. 10/347,155 entitled “Optical Discs Including Equi-Radial and/or Spiral Analysis Zones and Related Disc Drive Systems and Methods” filed on Jan. 15, 2003; U.S. patent application Ser. No. 10/347,119 entitled “Bio-Safe Dispenser and Optical Analysis Disc Assembly” filed Jan. 17, 2003; U.S. patent application Ser. No. 10/348,049 entitled “Multi-Purpose Optical Analysis Disc for Conducting Assays and Related Methods for Attaching Capture Agents” filed on Jan. 21, 2003; U.S. patent application Ser. No. 10/348,196 entitled “Processes for Manufacturing Optical Analysis Discs with Molded Microfluidic Structures and Discs Made According Thereto” filed on Jan. 21, 2003; U.S. patent application Ser. No. 10/351,604 entitled “Methods for Triggering Through Disc Grooves and Related Optical Analysis Discs and System” filed on Jan. 23, 2003; U.S. patent application Ser. No. 10/351,280 entitled “Bio-Safety Features for Optical Analysis Discs and Disc System Including Same” filed on Jan. 23, 2003; U.S. patent application Ser. No. 10/351,244 entitled “Manufacturing Processes for Making Optical Analysis Discs Including Successive Patterning Operations and Optical Discs Thereby Manufactured” filed on Jan. 24, 2003; U.S. patent application Ser. No. 10/353,777 entitled “Processes for Manufacturing Optical Analysis Discs with Molded Microfluidic Structures and Discs Made According Thereto” filed on Jan. 27, 2003; U.S. patent application Ser. No. 10/353,839 entitled “Method and Apparatus for Logical Triggering” filed on Jan. 28, 2003; and U.S. patent application Ser. No. 10/356,666 entitled “Methods For Synthesis of Bio-Active Nanoparticles and Nanocapsules For Use in Optical Bio-Disc Assays and Disc Assembly Including Same” filed Jan. 30, 2003. All of these applications are herein incorporated by reference in their entireties. They thus provide background and related disclosure as support hereof as if fully repeated herein.

Concluding Summary

All patents, provisional applications, patent applications, technical specifications, and other publications mentioned in this specification are incorporated herein in their entireties by reference.

While this invention has been described in detail with reference to certain preferred embodiments, it should be appreciated that the present invention is not limited to those precise embodiments. Rather, in view of the present optical bio-system disclosure that describes the current best mode for practicing the invention, many modifications and variations would present themselves to those of skill in the art without departing from the scope and spirit of this invention. The scope of the invention is, therefore, indicated by the following claims rather than by the foregoing description. All changes, modifications, and variations coming within the meaning and range of equivalency of the claims are to be considered within their scope.

Furthermore, those skilled in the art will recognize, or be able to ascertain, using no more than routine experimentation, many equivalents to the specific embodiments of the invention described herein.

1. A method of preparing a bio-disc having at least one analysis channel, the method comprising:
providing a membrane strip that is or can be dimensioned to fit within the at least one channel of the bio-disc;
applying one or more reagents on the membrane strip, wherein the membrane strip has a release efficiency that allows the one or more reagents to be released from the membrane strip into a solution placed in contact with the one or more reagents on the membrane strip; and
depositing the membrane strip in one of the at least one analysis channels of the bio-disc.
The method of claim 1, wherein the bio-disc comprises a semi-reflective layer having a thickness of less than about 400 Å.
The method of claim 1, wherein the bio-disc comprises a semi-reflective layer having a thickness of between about 100 and 300 Å.
The method of claim 1, wherein the bio-disc comprises a plurality of analysis channels and a plurality of membrane strips are deposited in the plurality of analysis channels.
The method of claim 1, wherein the plurality of reagents printed on the membrane strip are allowed to dry before the depositing step is performed.
The method of claim 1, wherein the membrane strip is a bibulous hydrophilic material.
The method of claim 1, wherein the membrane strip comprises hydrophilic polyethersulfone.
The method of claim 1, wherein the membrane comprises pores having a diameter equal to or greater than about 0.2 micrometers.
The method of claim 1, wherein the membrane strip has dimensions of about 3 mm by 5 mm by 0.3 millimeters.
The method of claim 1, wherein a volume of each of the plurality of reagents applied on the membrane strip is between about 2 and 5 microliters.
The method of claim 1, wherein the applying is performed using an automatic applicator.
The method of claim 1, wherein the applying is performed using a pipettor.
The method of claim 1, wherein the applying is performed using a printing device.
The method of claim 13, wherein the printing device comprises an ink jet printer.
An apparatus for quantifying an optical density change in colorimetric assays, the apparatus comprising:
an optical disc having one or more compounds deposited thereon, wherein the one or more compounds change one or more spectral characteristics in the presence of a target substance so that a spectral change by each of the one or more compounds is a function of a concentration of the target substance brought into contact with each of the one or more compounds;
optical elements configured to emit and direct radiation so that the radiation is incident on the compounds;
a detector configured to measure a value indicative of the spectral change for each of the one or more compounds;
a computing device configured to receive the value indicative of the spectral change from the detector and determine an amount or concentration of one or more target substances.
The apparatus of claim 15, wherein the detector comprises a spectrophotometer.
A method of determining a concentration of a sample, the method comprising:
introducing a liquid having a known concentration of a substance into a first fluidic channel in an optical disc;
placing a light blocking structure between a light source and a second fluidic channel;
determining a maximum light intensity by detecting an amount of light transmitted through the first fluidic channel;
determining a minimum light intensity by detecting an amount of light transmitted through the second fluidic channel; and
establishing a relationship between the minimum and maximum light intensities so that a concentration of a target substance present in a third fluidic channel may be determined based on an amount of light transmitted through the third fluidic channel and the relationship between the minimum and maximum light intensities.
The method of claim 17, wherein the relationship between the minimum and maximum light intensities is expressed as a ratio.
The method of claim 17, wherein the concentration of the target substance present in the third fluidic channel is greater than the known concentration of the substance in the first fluidic channel.
The method of claim 17, wherein the concentration of the target substance present in the third fluidic channel is less than the known concentration of the substance in the first fluidic channel.
The method of claim 17, wherein the relationship between the minimum and maximum light intensities is a function of the known concentration of the substance in the first fluidic channel.
The method of claim 17, further comprising introducing a liquid having a second known concentration of the substance, or a zero concentration of the substance, into a second fluidic channel in the optical disc;
A method of quantifying an amount of one or more analytes present in a biological sample, the method comprising:
providing an optical disc having one or more reagents located on or in one or more analysis zones of the optical disc;
introducing a sample onto or into the optical disc so that the sample contacts the one or more reagents on the optical disc;
incubating the optical disc for a period of time;
quantifying a spectral change in at least one portion of the disc resulting from introduction of the sample; and
determining an amount of the one or more analytes present in the sample based upon results from the quantifying step.
The method of claim 23, wherein the analyte is one of glucose and cholesterol.
The method of claim 23, wherein the analyte is a triglyceride.
The method of claim 23, wherein the depositing is performed by one or more of air evaporation, vacuum evaporation, enzyme immobilization, lyophilization, and reagent printing.
The method of claim 23, wherein the sample comprises one or more of a blood sample and a serum sample.
The method of claim 23, wherein the step of incubating is performed at about 37 degrees Celcius.
A method of quantifying an amount of one or more analytes present in a biological sample, the method comprising:
depositing one or more reagents onto respective one or more analysis zones on an optical disc;
applying a sample onto the optical disc so that the one or more reagents is brought into contact with the sample;
incubating the optical disc for a period of time;
emitting radiation having a know wavelength so that the radiation is incident upon the sample;
quantifying an amount of radiation transmitted through the sample in contact with each of the one or more reagents; and
determining an amount of the one or more analytes present in sample based upon results from the quantifying step.
